(19) United States
(12) Patent Application Publication
(10) Pub. No.: US 2023/0249003 A1
(43) Pub. Date: Aug. 10, 2023

(54) CONFORMAL PARTICLE THERAPY SYSTEM

(71) Applicant: ION BEAM APPLICATIONS SA, Louvain-la-Nueve (BE)

(72) Inventors: Rudi LABARBE, Louvain-la-Neuve (BE); Lucian HOTOIU, Louvain-la-Neuve (BE); Sebastien DE NEUTER, Louvain-la-Neuve (BE); Laurent COLLIGNON, Louvain-la-Neuve (BE); Arnaud PIN, Louvain-la-Neuve (BE)

(21) Appl. No.: 18/001,687

(22) PCT Filed: Jun. 24, 2021
(86) PCT No.: PCT/EP2021/067393

§ 371 Date: Dec. 13, 2022

(30) Foreign Application Priority Data
Jul. 3, 2020 (EP) 20184077.4

(51) Int. Cl. A61N 5/10

(52) U.S. Cl. CPC A61N 5/1077; A61N 5/1065

(57) ABSTRACT

A particle therapy system that is adapted to irradiate a target volume (1) with charged particles in compliance with a desired 3-D dose distribution. Such a desired 3-D dose distribution is achieved while delivering a plurality of particle energy distributions at the output of an energy-shaping device (10) crossed by an incident mono-energetic charged particle beam (6). The energy-shaping device comprises a plurality of groups (12, 22) of energy-shaping elements (11, 21), each of them comprising an individual layer of fluid or solid material (13), which thickness is adapted individually by a control unit (14). The use of configurable layers of fluids or solid materials makes the energy-shaping device reusable for treating different patients.

### FIELD OF THE INVENTION

The invention relates to a charged particle therapy system.

More particularly, the invention relates to a therapy system for irradiating a target volume within a patient with a charged particle beam, and comprising a charged particle beam generator, a beam transport system for transporting the charged particle beam, an irradiation device for delivering the charged particle beam to the target volume, and an energy-shaping device placed across a path of the charged particle beam.

The energy-shaping device comprises a plurality of energy-shaping elements that are each designed to modify the energy of incoming particles of a mono-energetic particle beam so that a mix of different particle energies are delivered at their output to form a Spread-Out Bragg Peak (SOBP) in a corresponding region of the target volume, with the aim that the irradiation of the target volume is more or less conform to its 3D shape.

### DESCRIPTION OF PRIOR ART

Charged particle therapy systems are well known in the art. Their function is to destroy unhealthy cells in a particular 3D region (hereafter “the target volume”) of a living being (hereafter “the patient”) by irradiating the target volume with a beam of charged particles such as a beam of protons, ions, etc. There currently exist several irradiation techniques for irradiating the target with the particle beam. These techniques can be roughly categorized into scattering techniques and scanning techniques. In the first category, a broad scattered beam irradiates the target volume as a whole, whereas in the second category a narrow beam irradiates the target volume while scanned over it.

Whatever the irradiation technique, an aim has always been to reduce unwanted irradiation of cells of the patient lying outside of the target volume, both laterally (X,Y) and in depth (Z). This aim is often referred to as “improving conformal irradiation”.

With a view to improving conformal irradiation, particularly in the depth direction, several solutions have been proposed, such as the placement of an energy-shaping device (sometimes also called an energy modulator) in the path of the particle beam (e.g. ridge filters, range compensators, energy selection system).

An example of a therapy system comprising such an energy modulator is disclosed in American patent application US2018068753A1. According to such a known system, the energy modulator (called a “ridge filter” in document US2018068753A1) is placed across the beam path between the charged particle beam generator and the patient. A beam spreading device (sometimes called a “scatterer”), located upstream of the energy modulator, spreads the particle beam over the surface of the energy modulator. The energy modulator is made up of a plurality of damping elements, each damping element having a cross-sectional area that changes stepwise along the irradiating direction. When charged particles pass through such a damping element, a specific distribution of particle energies is generated at the output of the damping element, and this specific energy distribution will result in a corresponding specific Spread-Out Bragg Peak profile (SOBP) in the crossed region of the target volume when it is irradiated by the particle beam through the damping element. As is well known, the distribution of particle energies at the output of a damping element will depend on the material and the geometry of the damping element, more particularly on the various widths and heights of its staircase steps. The height of a staircase step will determine the mean particle energy at its output, whereas the width of a staircase step will determine a particle ratio.

Such a known energy modulator is made bespoke to a given patient and to a specific field to be irradiated and hence cannot be reused for another patient or for another beam orientation.

Another example of a known therapy system comprising such an energy modulator is disclosed in Korean patent number KR101546656. According to such a known system, the energy modulator (called a “variable compensator” in document KR101546656) is made up of a plurality of damping elements, each damping element comprising a column of fluid of a certain height extending in the irradiating direction. When charged particles pass through such a damping element, their energy decreases, thereby decreasing the corresponding depth of the Bragg peak in the target volume in relation to the height of fluid in the column.

Furthermore the height of the column of fluid of each damping element is individually controlled by a control unit, allowing for adapting the penetration depth of the charged particles of the irradiating beam to the distal edge of the target volume. Such a known particle therapy system is however not adapted to achieve an SOBP in the target volume.

Another example of a known therapy system comprising such an energy modulator is disclosed in American patent publication number U52008/0260098. Such a known energy modulator is similar to the modulator of KR101546656 and is therefore also meant to modulate the depths of the Bragg peaks in order to conform only to the distal edge of the target volume. It is not capable or at least not configured to deliver patient-specific and planned SOBPs to each 3D region of the target volume when the beam is irradiated according to a single main beam direction to the target. Eventually, SOBPs can be generated with such system by irradiating the target according to various main beam directions while changing the damping power of the various damping elements at a plurality of irradiation angles, though it is not clear from this document how this could concretely be achieved. In any case, having to change the main beam direction and having to change the damping power of the various damping elements in the course of a treatment increases the treatment time, which is not desirable. Furthermore, such approach does not permit to deliver 3D conformal doses to the target with enough degrees of freedom because of the interdependency of the doses delivered at the various irradiation angles.

### SUMMARY OF THE INVENTION

It is therefore an object of the invention to provide a therapy system that is adapted to irradiate the target volume in better compliance with a desired 3-D dose distribution in the target volume and whose energy modulator can be reused or reconfigured for different patients and/or for different irradiation fields.

To this end, the invention provides a therapy system for irradiating a target volume within a patient with a charged particle beam, comprising:
a charged particle beam generator,
a beam transport system for transporting the charged particle beam,
an irradiation device for delivering the charged particle beam to the target volume,
an energy-shaping device placed across a path of the charged particle beam, said energy-shaping device comprising a first pre-defined group of neighbouring energy-shaping elements that is adapted to deliver a first desired particle energy distribution at an output of said first pre-defined group of energy-shaping elements when crossed by particles of the charged particle beam and at least a second pre-defined group of neighbouring energy-shaping elements which is adapted to deliver a second desired particle energy distribution at an output of said second pre-defined group of energy-shaping elements when crossed by particles of the charged particle beam, said second desired particle energy distribution being different from said first desired particle energy distribution.

Each energy-shaping element of each of the first and second pre-defined groups of energy-shaping elements comprises an individual layer of fluid or of a solid material.

The therapy system further comprises a control unit which is configured:
to adjust the thickness of each fluid or solid material of each individual layer of fluid or of solid material of the energy-shaping elements of the first pre-defined group of neighbouring energy-shaping elements to obtain said first desired particle energy distribution when the irradiation device is oriented to deliver the particle beam to the target volume according to a first main beam direction, and
to adjust the thickness of each fluid or solid material of each individual layer of fluid or of solid material of the energy-shaping elements of the second pre-defined group of neighbouring energy-shaping elements to obtain said second desired particle energy distribution when the irradiation device is oriented to deliver the particle beam to the target volume according to the first main beam direction, the said thickness of each fluid or of solid material being a thickness in a propagation direction of the charged particles of the charged particle beam.

In the context of the present invention, a “particle energy distribution at the output of a group of energy-shaping elements” is generally to be understood as a probability density function of particle energies, which function gives, for each particle energy value, the ratio of the number of particles having said particle energy value at the output of the group of energy-shaping elements to the total number of particles at the output of the group of energy-shaping elements.

In the context of the present invention, a pre-defined group of neighbouring energy-shaping elements means that such groups are not to be considered as “any group of energy-shaping elements” but are rather groups of neighbouring energy-shaping elements which are well defined and well known in advance to the control unit. A pre-defined group of neighbouring energy-shaping elements generally correspond to a specific pre-defined region of the target volume into which a desired or planned dose distribution and hence a desired or planned SOBP is to be achieved when that specific pre-defined region of the target is irradiated with the charged particle beam after particles of the charged particle beam have crossed the energy-shaping elements of said pre-defined group. The said desired or planned dose distribution may for example come from a Treatment Planning System.

Unlike the system disclosed in document US2018068753A1, a therapy system according to the invention can be re-used for different target volumes by adjusting the thicknesses of the fluids or of the solid materials according to the specific target volume to be treated.

Unlike the invention disclosed in document KR101546656 a therapy system according to the invention is adapted to generate specifically planned SOBPs in the various 3D regions of the target volume and is capable of a better conformal irradiation with a single and configurable device.

Unlike the system disclosed in document U52008/0260098 a therapy system according to the invention is adapted to generate specifically planned SOBPs in the various 3D regions of the target volume while the beam is directed to the target volume according to a single main beam direction and is hence faster and more accurate.

Preferably, the control unit is configured such that:
the first desired particle energy distribution comprises a first particle ratio (PRmin1) at a first minimum energy (Emin1) and a second particle ratio (PRmax1) at a first maximum energy (Emax1),
the second desired particle energy distribution comprises a third particle ratio (PRmin2) at a second minimum energy (Emin2) and a fourth particle ratio (PRmax2) at a second maximum energy (Emax2),
   and such that Emax1 is different from Emax2

With such a preferred therapy system, a better irradiation conformity to the distal edge of the target volume can be achieved.

More preferably the control unit is configured such that PRmax1 is different from PRmax2. With such a preferred therapy system, an even better irradiation conformity to the target volume can be achieved.

Preferably the control unit is configured such that Emin1 is different from Emin2. With such a preferred therapy system, a better irradiation conformity to the proximal edge of the target volume can be achieved.

Even more preferably the control unit is configured such that PRmin1 is different from PRmin2. With such a preferred therapy system, an even better irradiation conformity to the target volume can be achieved.

Preferably the control unit is configured such that (Emax1-Emin1) is different from (Emax2−Emin2). With such a preferred therapy system, an even better irradiation conformity to the target volume can be achieved.

Preferably, each energy-shaping element has a cylindrical surface. With such a preferred therapy system, the energy-shaping elements can be aligned close to each other, so saving space and increasing compaction.

More preferably, all energy-shaping elements have the same hexagonal cross section, which allows for a most compact energy-shaping device.

Preferably, each energy-shaping element is a tube containing a fluid or a solid material. With such a preferred therapy system, the thickness of each layer of fluid or solid material can be easily adjusted by the control unit. Also, different energy-shaping elements can hold different fluids or solid materials with different stopping powers.

Preferably, the said fluid is a liquid. Exemplary liquids are furan (C₄H₄O) and solutions of glucose (C₆H₁₂O₆).

Preferably, the said solid material is a granular solid material.

Preferably, the energy-shaping elements are aligned with a propagation direction of the particles of the charged particle beam that cross them.

More preferably, each group of energy-shaping elements is aligned with respect to a propagation direction of the particles of the incident charged particle beam.

Preferably, the therapy system comprises a beam scanner to scan the charged particle beam over the target volume, and a spot size of the charged particle beam in front of the energy-shaping device is substantially equal to the cross section of the first pre-defined group of neighbouring energy-shaping elements and substantially equal to the cross section of the second pre-defined group of neighbouring energy-shaping elements.

Alternatively, the energy-shaping elements are arranged transversely with respect to a propagation direction of the particles of the charged particle beam, preferably perpendicularly with respect to a propagation direction of the particles of the charged particle beam. With such an alternative, energy-shaping elements can pile up across a propagation direction of the particles and the number of piled layers of energy-shaping elements, their respective orientations, their respective heights and cross-sections as well as the fluids or solid materials they contain can be adapted to achieve a desired SOBP in the target volume.

Preferably the charged particle beam generator is a cyclotron or a synchrotron. Preferably a nominal beam energy at an output of the charged particle beam generator is in the range of 70 MeV to 250 MeV.

### SHORT DESCRIPTION OF THE DRAWINGS

These and further aspects of the invention will be explained in greater detail by way of examples and with reference to the accompanying drawings in which:

FIG. 1 shows a schematic view of a therapy system according to the invention;

FIG. 2a shows a 3-D view of an exemplary energy-shaping device of a therapy system according to the invention;

FIG. 2b shows a sectional view of the energy-shaping device of FIG. 2a;

FIG. 2c shows a sectional view of a preferred energy-shaping device of a therapy system according to the invention;

FIG. 3a shows a more detailed view of the therapy system of FIG. 1, when in operation;

FIG. 3b shows exemplary dose distributions along various beam directions in an XZ plane when using the therapy system of FIG. 3a;

FIG. 3c shows exemplary particle energy distributions along various beam directions in said XZ plane when using the therapy system of FIG. 3a;

FIG. 4 shows groups of energy shaping elements according to the invention where the energy-shaping elements are tubes aligned with the propagation direction of the particles of the charged particle beam;

FIG. 5a shows groups of energy shaping elements according to the invention where the energy-shaping elements are tubes arranged transversely with respect to the propagation direction of the particles of the charged particle beam;

FIG. 5b shows a zoom on neighbouring energy-shaping elements of FIG. 5a;

FIG. 6 shows energy-shaping elements arranged as in FIG. 5a, but filled with solid materials instead of liquids.

Unless otherwise indicated, the figures are not drawn to scale. Generally, identical components are denoted by the same reference numerals in the figures.

### DETAILED DESCRIPTION OF PREFERRED EMBODIMENTS

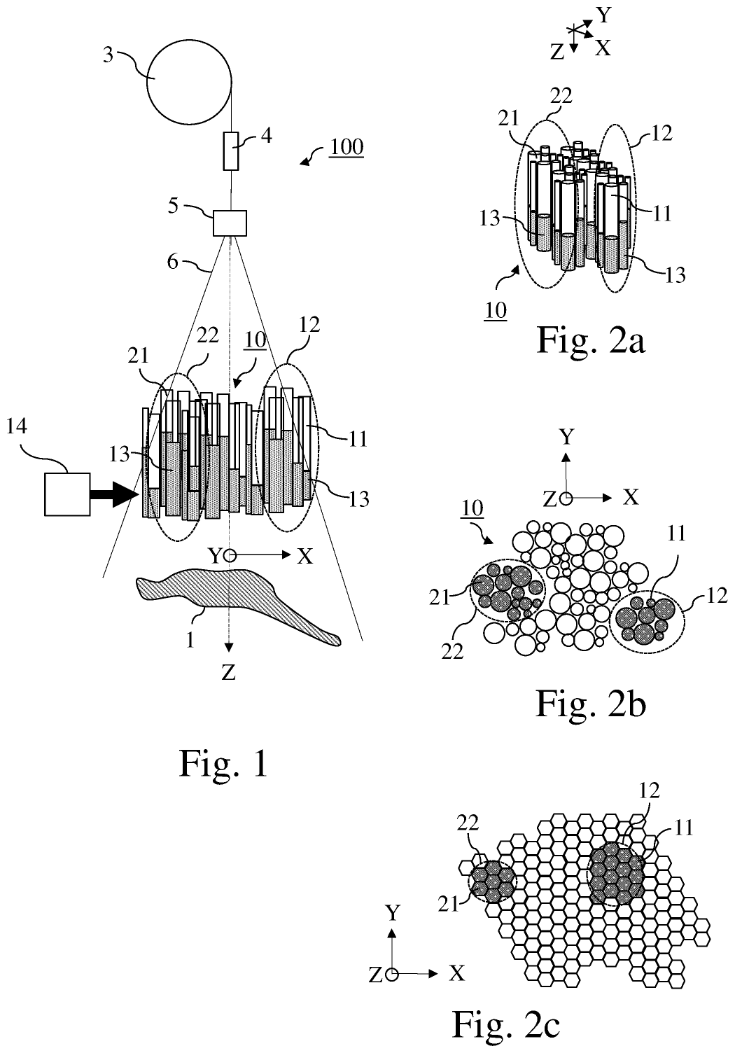

FIG. 1 shows a schematic view of an exemplary therapy system (100) according to the invention. The system comprises a charged particle beam generator (3) (such as a cyclotron or a synchrotron for example) for generating a typically mono-energetic beam of charged particles, such as protons or carbon ions or any other type of ion. A typical beam energy delivered by the charged particle beam generator (3) is for example in the range of 70 MeV to 250 MeV. The system also comprises a beam transport system (4) for transporting the charged particle beam from the particle beam generator (3) to an irradiation device (5) (sometimes called a nozzle). The irradiation device (5) has a main beam axis (Z) (also called main beam direction) and is adapted for delivering the charged particle beam (6) in an appropriate form to a target volume (1) within a patient (the patient not shown here). The system also comprises an energy-shaping device (10) which is placed in the beam path between the generator (3) and the target volume (1). In this example, the energy-shaping device (10) is placed in the beam path between the irradiation device (5) and the patient but it may also be integrated into the irradiation device (5).

Such a therapy system may apply various target irradiation techniques such as beam scattering, beam wobbling, beam scanning, or other methods. The energy-shaping device (10) is placed downstream of the device performing the said beam scattering, beam wobbling or beam scanning. The irradiation device (5) may be mounted on a gantry for rotation of said device about an isocenter or it may be of the fixed beam line type or of any other type. Such systems are well known in the art and will therefore not be described in further detail.

Of interest here is the energy-shaping device (10) which comprises a first pre-defined group (12) of neighbouring energy-shaping elements (11) that is adapted to deliver a first desired particle energy distribution at an output of said first pre-defined group (12) of energy-shaping elements when crossed by particles of the charged particle beam (6) and at least a second pre-defined group (22) of neighbouring energy-shaping elements (21) which is adapted to deliver a second desired particle energy distribution at an output of said second pre-defined group (22) of energy-shaping elements when crossed by particles of the charged particle beam, said second desired particle energy distribution being different from said first desired particle energy distribution.

In this example, each energy-shaping element comprises an individual layer of fluid (13), or of a solid material, having a thickness. Preferably, the said fluid is a liquid. Exemplary liquids are furan (C₄H₄O) and solutions of glucose (C₆H₁₂O₆). Preferably, the solid material is a granular material or a material in the form of powder. Exemplary granular solid materials are granules of polymethyl methacrylate (PMMA), granules of polystyrene, granules of Lexan, granules of high-density polyethylene.

The system further comprises a control unit (14) which is configured:
to adjust the thickness of each fluid or solid material of each individual layer of fluid or solid material (13) of the energy-shaping elements (11) of the first pre-defined group (12) to obtain said first desired particle energy distribution when the irradiation device is oriented to deliver the particle beam to the target volume according to a first main beam direction (the Z direction on FIG. 1), and
to adjust the thickness of each fluid or solid material of each individual layer of fluid or solid material (13) of the energy-shaping elements (21) of the second pre-defined group (22) to obtain said second desired particle energy distribution when the irradiation device is oriented to deliver the particle beam to the target volume according to the first main beam direction (the same Z direction on FIG. 1).

In the context of the present invention, the said thickness of each fluid or solid material, is a thickness of said fluid or solid material in a propagation direction of the charged particles.

The thickness of each fluid or solid material of each individual layer of fluid or solid material of the first pre-defined group of energy-shaping elements is adjusted by the control unit according to a first desired spatial dose distribution in that first region of the target volume (1) which will be irradiated by the charged particles outputting the first pre-defined group of energy-shaping elements. The said desired first spatial dose distribution is for example a dose distribution as prescribed by a treatment plan for the said first region of the concerned target volume (1).

The thickness of each fluid or solid material of each individual layer of fluid or solid material of the second pre-defined group of energy-shaping elements is adjusted by the control unit according to a second desired spatial dose distribution in that second region of the target volume (1) which will be irradiated by the charged particles outputting the second pre-defined group of energy-shaping elements. The said desired second spatial dose distribution is for example a dose distribution as prescribed by the treatment plan for the said second region of the concerned target volume (1).

Preferably, the control unit (14) adjusts the thickness of each fluid or solid material, of each individual layer of fluid or solid material of the first and second pre-defined groups of energy-shaping elements before the particle beam (6) is turned on.

In the example of FIG. 1, the energy-shaping elements (11, 21) of the first and second pre-defined groups (12, 22) are cylindrical tubes, oriented in the Z direction, and are at least partially filled with liquids or with a solid material such as a granular solid material for example. However any other embodiment that would put layers of fluids or solid materials across a path of the charged particles would fit as long as the thicknesses (in the propagation direction of the charged particles) of such layers of fluids or of solid materials are adjusted by means of the control unit (14) with a view to achieving the desired particle energy distribution at the output of the first pre-defined group (12) of neighbouring energy-shaping elements (11) and at the output of the second pre-defined group (22) of neighbouring energy-shaping elements (21) while the irradiation device is oriented to deliver the particle beam to the target volume according to the first main beam direction (the Z direction on FIG. 1).

With such cylindrical tubes oriented in the Z-direction (or according to a propagation direction of the charged particles that cross the said tubes) as energy-shaping elements, the thickness of liquid of a particular tube can for example be adjusted by using a first piston placed inside the tube to separate the liquid from a gas such as air for example. In this example, the first piston will move in the tube according to the pressures of the liquid and gas on both sides of the first piston until equilibrium of their respective pressures is achieved. The liquid and the gas may each be held in a dedicated tank, each tank being fluidly connected respectively to opposite ends of the tube, wherein their respective pressures are adjusted by the control unit (14), for example by moving a second piston in the liquid tank. The piston in the liquid tank can for example be moved back and forth by means of a stepper motor acting on the second piston via a shaft, each step of the motor eventually translating into a variation of liquid thickness in the tube. The liquid tank may for example be a syringe, the stepper motor being connected to the piston of the syringe. The connection between an end of the tube and a tank is adapted to have the tank out of the path of the particle beam. The said connection can for example be shaped as an elbow with a 90° bend and have sufficient length to arrange the tank out of the path of the particle beam. In this example all the tubes are equipped in the same way, the lengths of the various connections being adapted to accommodate the number of tubes, and their stepper motors are each controlled individually by the control unit (14). A similar system can be used to adjust the thickness of a granular solid material in a tube instead of a liquid.

FIG. 2a shows a 3-D view of the energy-shaping device (10) of FIG. 1, and FIG. 2b shows a cross-section of the energy-shaping device (10) of FIG. 1 in a plane (XY) that is perpendicular to the main beam axis (Z). Those figures illustrate the first pre-defined group (12) of neighbouring energy-shaping elements (11) and the second pre-defined group (22) of neighbouring energy-shaping elements (21). In those figures the energy-shaping elements (11, 21) are tubes with various cross-sections and aligned with the Z axis. The number of such tubes in each group (12, 22) of neighbouring energy-shaping elements and their respective cross-sections are chosen according to the particle energy distribution to be achieved at the output of that group of neighbouring energy-shaping elements.

The number of tubes belonging to a given group (12, 22) of neighbouring energy-shaping elements and their respective cross-sections must be chosen so as to achieve a desired SOBP between the frontal and distal edges of the target volume (1) along the path of the charged particles that will output that given group of neighbouring energy-shaping elements.

In the exemplary case where each tube of a given group of energy-shaping elements is filled by the control unit with a different thickness of a same liquid or of a same solid material, each tube of that given group of neighbouring energy-shaping elements will output charged particles of a different energy, each energy being at the origin of a particular Bragg curve (and then Bragg Peak) of the desired SOBP in the target volume. The fraction of charged particles of a particular energy that will output that first pre-defined group (12) of neighbouring energy-shaping elements is approximately proportional to the cross-section of the tube belonging to that given group (12) of neighbouring energy-shaping elements which fluid or solid material thickness has been adjusted by the control unit (14) to output charged particles of that particular energy. The control unit (14) turns the desired/planned particle energy distribution at the output of a given group of neighbouring energy-shaping elements into individual liquid or solid material thicknesses and fills the various energy-shaping elements of that group accordingly.

The number of tubes in the first or second pre-defined groups of energy-shaping elements and their respective sections must also comply with the diameter of a corresponding cylindrical sub-volume to be irradiated in the target volume, as defined for example by the said treatment plan (spatial dose distribution). Indeed the overall cross-section of the first pre-defined group (12) of energy-shaping elements must fit, as much as possible, the cross section of the said corresponding cylindrical sub-volume in the target volume. The same holds of course for the second pre-defined group of neighbouring energy-shaping elements (22).

In the case of pencil beam scanning (PBS), the overall cross-section of the first pre-defined group (11) of neighbouring energy-shaping elements must also fit, as much as possible, the size and shape of the PBS spot at the input of said first pre-defined group (11) of neighbouring energy-shaping elements. The same holds of course for the second pre-defined group (22) of neighbouring energy-shaping elements.

In these examples, each tube (11, 21) has for example a diameter comprised between 2 mm and 10 mm, the first pre-defined group of tubes comprises for example between 5 and 15 tubes (11), and the second pre-defined group of tubes comprises for example between 5 and 15 tubes (21).

FIG. 2c shows a sectional view in the XY plane of a preferred energy-shaping device of a therapy system according to the invention. In such a preferred embodiment, all energy-shaping elements (11, 21) are tubes of the same hexagonal section arranged in a honeycomb fashion.

Such an energy-shaping device (10) is specially designed to reduce the energy of incident charged particles, so that a desired particle energy distribution will be present at the output of a pre-defined group of neighbouring energy-shaping elements.

When the charged particles outputting a given group of neighbouring energy-shaping elements enter the target volume (1), several Bragg Peaks are generated in a corresponding region of the target volume (1), the combination of which will result in a so called “Spread Out Bragg Peak” (SOBP). In themselves, the function and basic operation of such an energy shaping device are well known in the art and will therefore also not be described further.

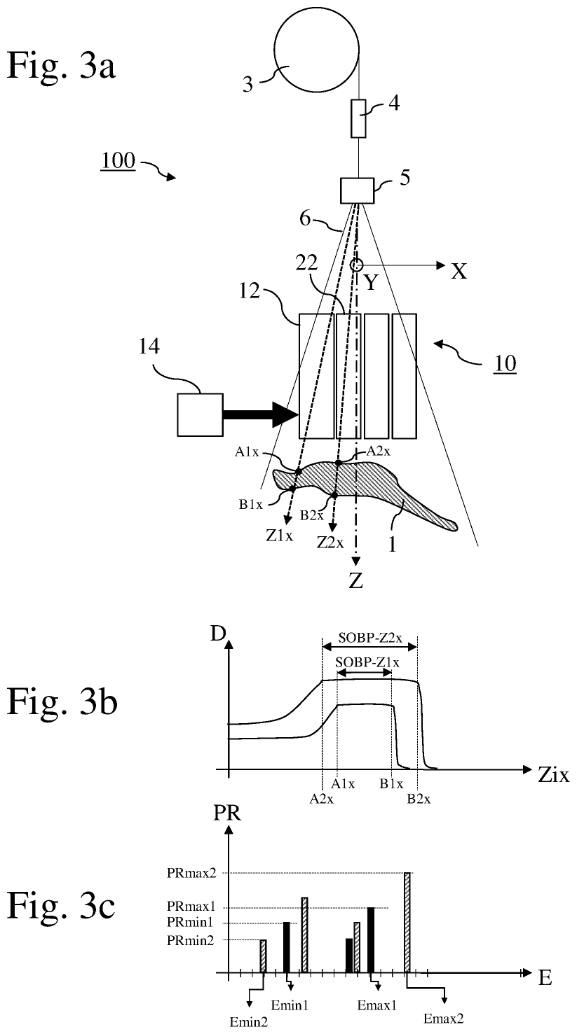

FIG. 3a shows a view of the therapy system (100) of FIG. 1 when in operation, namely after the control unit (14) has adjusted the thickness of each fluid or solid material of each individual layer of fluid or solid material of the energy-shaping elements of the first pre-defined group (12) to obtain said first desired particle energy distribution and has adjusted the thickness of each fluid or solid material of each individual layer of fluid or solid material of the energy-shaping elements of the second pre-defined group (22) to obtain said second desired particle energy distribution, and while the charged particle beam is irradiating the target volume (1) according to the first direction (the Z direction on FIG. 3a).

FIG. 3a more particularly shows a cross-section of a particular target volume (1) in an XZ plane, as well as a corresponding cross-section of the energy-shaping device (10) in the same XZ plane. In this XZ plane, charged particles of the particle beam (6) may for instance follow a first beam direction (Z1x) intercepting a first region of the target volume (1) delimited in depth by two first points (A1x, B1x). These charged particles cross the first pre-defined group (12) of neighbouring energy-shaping elements (11) and produce in the target volume (1) a first SOBP (SOBP-Z1x) which profile (essentially width, height and depth position) substantially corresponds to a desired dose distribution in said first region when these charged particles follow the first beam direction (Z1x). The desired dose profile along the first beam direction (Z1x) as well as the desired first SOBP (SOBP-Z1x) is shown in the graph of FIG. 3b in which the horizontal axis Z1x represents a beam direction such as Z1x or Z2x. The same holds for charged particles following the second beam direction (Z2x).

The corresponding desired first distribution of particle energies to be produced at the output of the first pre-defined group (12) of neighbouring energy-shaping elements (11) is shown in FIG. 3c wherein the horizontal axis indicates particle energies (E), represented by their mean values within ranges, on a linearly graduated scale (tick marks) and wherein the vertical axis indicates the ratio (PR) of the number of particles having a mean particle energy at the output of the first pre-defined group (12) of neighbouring energy-shaping elements (11) to the total number of particles crossing the first pre-defined group (12) of neighbouring energy-shaping elements (11).

As shown on FIG. 3c, said first energy distribution comprises a first particle ratio (PRmin1) at a first minimum energy (Emin1) and a second particle ratio (PRmax1) at a first maximum energy (Emax1). The first minimum energy (Emin1) and the first maximum energy (Emax1) respectively correspond to the depth of first point (A1x) and to the depth of the second point (B1x) in the target volume (1).

From this desired first distribution of particle energies, the specific thicknesses of the layers of fluids or solid material of the first pre-defined group (12) of neighbouring energy-shaping elements (11) can be computed according to known methods and then set by the control unit before irradiation of the target volume is started.

FIGS. 3a, 3b and 3c also show a second pre-defined group (22) of neighbouring energy-shaping elements (21), as well as a corresponding desired dose profile and SOBP (SOBP-Z2x)) and a desired second desired particle energy distribution when the −particles of the particle beam (6) follow a second beam direction (Z2x) in the XZ plane. As can be seen on FIG. 3c, the second desired particle energy distribution comprises a third particle ratio (PRmin2) at a second minimum energy (Emin2) and a fourth particle ratio (PRmax2) at a second maximum energy (Emax2). The second minimum energy (Emin2) and the second maximum energy (Emax2) respectively correspond to the depth of another first point (A2x) and to the depth of another second point (B2x) in the target volume (1).

From this desired second distribution of particle energies, the specific thicknesses of the layers of fluids or solid materials of the second pre-defined group (22) of neighbouring energy-shaping elements can also be computed according to known methods and then set by the control unit before irradiation of the target volume is started.

As will moreover be understood, the filtering effect of several neighbouring energy-shaping elements filled with a same height of the same fluid or solid material is more or less equivalent to the filtering effect of a single energy-shaping element of a larger cross-section (i.e. the cross-section multiplied by the number of tubes) filled with that same height of that same fluid or solid material.

As one can see on FIG. 3c, the control unit is preferably configured to adjust the thickness of each fluid or solid material of each individual layer of fluid or solid material of the energy-shaping elements (11) of the first pre-defined group (12) and to adjust the thickness of each fluid or solid material of each individual layer of fluid or solid material of the energy-shaping elements of the second pre-defined group (22)such that Emax1 is different from Emax2, preferably also such that PRmax1 is different from PRmax2, preferably also such that Emin1 is different from Emin2, preferably also such that PRmin1 is different from PRmin2, preferably also such that (Emax1−Emin1) is different from (Emax2−Emin2). With such capabilities, a good conformal irradiation of target volume can be obtained.

Such desired particle energy distributions can be achieved, for example, while scanning the particle beam (6) over the energy-shaping device (10) after the control unit (14) has adjusted the thickness of each fluid or solid material of each individual layer of fluid or solid material of the energy-shaping elements to achieve said desired particle energy distributions.

In such a case, the therapy system preferably comprises a beam scanner to scan the charged particle beam over the energy-shaping device. Such a beam scanner is well known in the art and may for example comprise electromagnets placed around the beam line for deviating the particle beam (6) in the X and Y directions. Hence, when scanning a particle beam (6) having for example a fixed energy over the energy-shaping device (10), a good depth-conformal irradiation of the target volume can be achieved, preferably in a single scan, namely a scan wherein the particle beam passes only once over each pre-defined group of energy-shaping elements.

In case the therapy system scans the beam, such as when using the known Pencil Beam Scanning (PBS) technique for example, the energy-shaping elements are sized such that a spot size of the charged particle beam (6) in front of the energy-shaping device (10) is substantially equal to the cross section of the first pre-defined group (12) of neighbouring energy-shaping elements (11) and substantially equal to the cross section of the second pre-defined group (22) of neighbouring energy-shaping elements (21).

Such desired particle energy distributions can also be achieved with single or double scattering of the charged particle beam before it reaches the energy-shaping device. In such an embodiment the beam is scattered so that substantially all the pre-defined groups of energy-shaping elements are crossed by scattered charged particles. A final collimator may optionally be used to ensure that the scattered beam is conformant to the lateral border of the target volume (1).

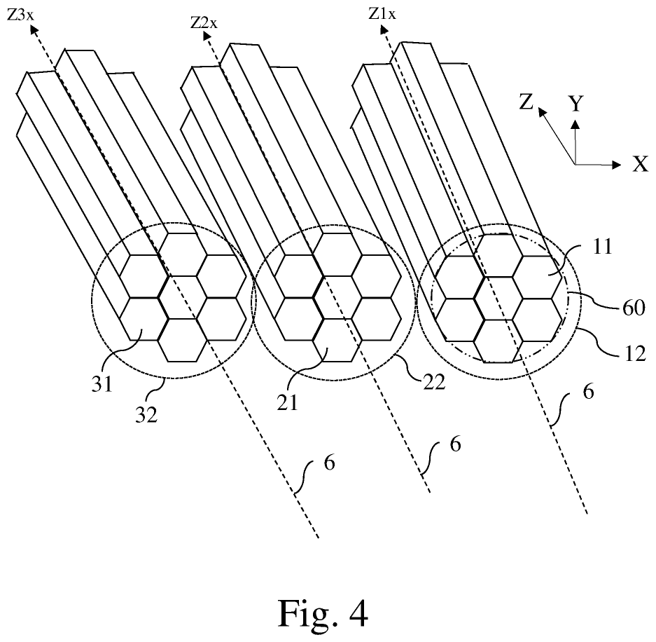

FIG. 4 shows three pre-defined groups (12, 22, 32) of neighbouring energy-shaping elements (11, 21, 31) according to an exemplary embodiment of, the invention, wherein each pre-defined group (12, 22, 32) of energy-shaping elements is aligned with respect to a propagation direction (Z1x, Z2x, Z3x) of the particles of the incident particle beam (6). Preferably, all energy-shaping elements of a given pre-defined group (12, 22, 32) are aligned with the propagation direction (Z1x, Z2x, Z3x) of the incident particle beam (6). With such a preferred embodiment an incident charged particle will only cross a single energy-shaping element. Such an embodiment can for example be used in conjunction with a scanning irradiation method where each pre-defined group (12, 22, 32) of neighbouring energy-shaping elements is respectively positioned in and aligned with the propagation direction (Z1x, Z2x, Z3x) of an incident scanned beam.

In the case of pencil beam scanning (PBS), and as shown on FIG. 4, the overall cross-section of each pre-defined group of neighbouring energy-shaping elements (12) must preferably fit, as much as possible, the size of the PBS spot (60) at the input of said each pre-defined group of neighbouring energy-shaping elements.

FIG. 4 illustrates energy-shaping elements that are tubes of hexagonal section. However the sections of the tubes can be of any shape, and their respective sections can vary.

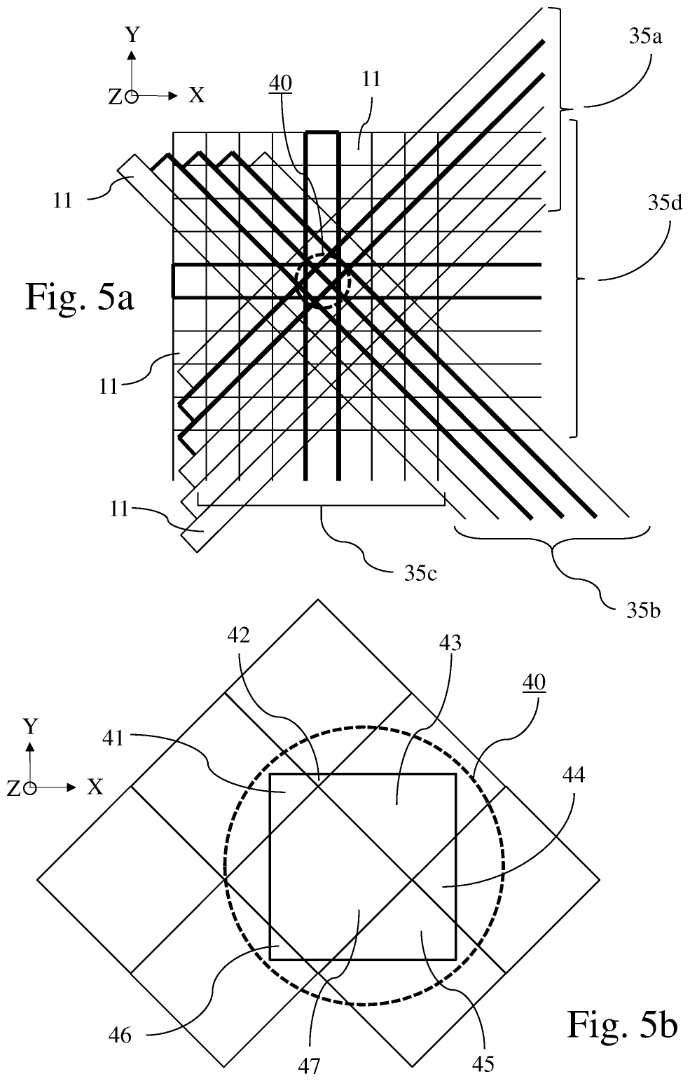

FIG. 5a shows an alternative embodiment of a therapy system (100) according to the invention. It is similar to the therapy system described hereinabove, except that the energy-shaping elements (11) are here arranged transversely with respect to a propagation direction of the particles of the charged particle beam, preferably perpendicularly with respect to a propagation direction of the particles of the charged particle beam, as shown on FIG. 5a with an XYZ referential, Z being the main beam direction.

In this example, the energy-shaping elements (11) are tubes of rectangular sections arranged side-by-side in piled layers, each layer being in a plane perpendicular to the main propagation direction (Z) of the particles of the charged particle beam (in FIG. 5a the main propagation direction (Z) of the particle beam is perpendicular or oblique to the plane of the sheet). Each layer of tubes has a different height and also a different orientation in that plane. FIG. 5a illustrates an embodiment that comprises four layers (35a, 35b, 35c, 35d), but any number of layers is possible. Also the sections of the tubes may have another shape than rectangular. Each tube (11) can be filled with a fluid, preferably a liquid such as furan (C₄H₄O) or solutions of glucose (C₆H₁₂O₆) for example, or with a solid material such as a granular solid material for example, or left empty, individually, by the control unit (14) and according to the same or similar criteria as described hereinabove. Also the types of fluid or of solid material can differ for each tube. According to such an embodiment, a pre-defined group of neighbouring energy-shaping elements comprises one or more tubes from a plurality of layers. FIG. 5a shows such an exemplary selection of neighbouring energy-shaping elements of a first pre-defined group, highlighted by their bolded borders. The layers of tubes are oriented and positioned so that the neighbouring energy-shaping elements define piles of fluids or of solid material of different sections, highlighted with the dashed circle (40) in FIG. 5a. Those sections are illustrated in FIG. 5b where we can see seven piles of fluids or of solid material (41 to 47), each with a different section and also a different stopping power. In case the therapy system comprises a scanner to scan the particle beam over the energy-shaping device (10), the area of the dashed circle (40) preferably corresponds substantially to the spot size of the particle beam at this location.

More generally, the energy-shaping elements of a pre-defined group are positioned individually with a view to defining various piles of layers of fluids or of solid material along the paths of incident charged particles, each fluid or solid material featuring a possibly different stopping power, and each pile featuring a possibly different area intersecting the incident charged particle beam. So each pile of layers of fluids or of solid material outputs particles of a given energy (or energies within a range which width is similar to the width of the range of incident particle beam) which will result from the different thicknesses and stopping powers of the layers of fluids or of solid material making the pile, while the fraction of the incident charged particles that will have that given energy (or energies) will depend on the intersected area of the pile of layers of fluids or of solid material (i.e. the area intersecting the charged particles of the incident charged particle beam). For the embodiment illustrated in FIGS. 5a and 5b, each pile of layers of fluids or of solid material is made of a tube while the intersecting areas are the sections of the tubes. In case the energy-shaping elements are arranged transversally with respect to a propagation direction of the particles of the charged particle beam, the energy-shaping elements (11) may alternatively be plain rods of solid material instead of tubes filled with fluids or with solid materials.

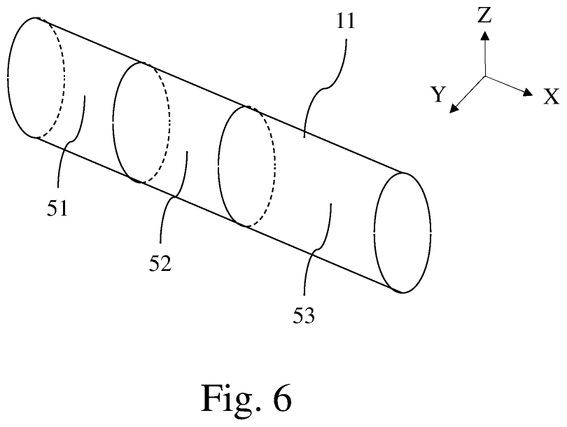

What is discussed above and illustrated in FIG. 5a and FIG. 5b therefore also applies if the energy-shaping elements are plain rods of solid materials. The geometrical considerations remain valid and the stopping powers of the piles of layers of solid materials can be adapted thanks to an appropriate choice of the involved solid materials. Such solid materials can for example be different types of plastic such as polymethyl methacrylate (PMMA), polystyrene, Lexan, high density polyethylene or metals such as brass or tungsten. Unlike fluids, solid materials offer the possibility to mix several materials in any single energy-shaping element. More precisely we can have various solid materials next to each other contained in an energy-shaping element (11) as illustrated in FIG. 6, where such an energy-shaping element (11) is a hollow tube. In the example of FIG. 6 there are three different solid materials (51, 52, 53), each of them occupying a portion of the hollow tube (11). In particular, each energy-shaping element (11) could include an individual number of solid materials (51, 52, 53), and the locations of the borders between the various solid materials could be chosen individually too. Such a configuration increases the number of degrees of freedom to achieve conformal irradiation. Energy-shaping elements made of plain rods of solid materials can be moved by the control unit in an appropriate treatment configuration in a way that is for example similar to multi-leaf collimators, i.e. by means of stepper motors moving the rods transversally back and forth in their respective treatment positions.

Energy-shaping elements made of tubes filled with solid materials can be arranged in an appropriate treatment configuration by the control unit, for example by controlling stepper motors pushing rods of solid materials in the tubes from one end or from the other end of each tube.

The present invention has been described in terms of specific embodiments, which are illustrative of the invention and not to be construed as limiting. Reference numerals in the claims do not limit their protective scope. Use of the verbs “to comprise”, “to include”, “to be composed of”, or any other variant, as well as their respective conjugations, does not exclude the presence of elements other than those stated. Use of the article “a”, “an” or “the” preceding an element does not exclude the presence of a plurality of such elements.

The invention may also be described as follows: a particle therapy system that is adapted to irradiate a target volume (1) with charged particles in compliance with a desired 3-D dose distribution. Such a desired 3-D dose distribution is achieved while delivering a plurality of particle energy distributions at the output of an energy-shaping device (10) crossed by an incident mono-energetic charged particle beam (6). The energy-shaping device comprises a plurality of pre-defined groups (12, 22) of energy-shaping elements (11, 21), each energy-shaping element of each group comprising an individual layer of fluid or of solid material (13), which thickness is adapted individually by a control unit (14) prior to irradiation in order to obtain said desired 3-D dose distribution while the target volume is thereafter irradiated according to a single main beam direction (Z).

1. Therapy system for irradiating a target volume within a patient with a charged particle beam, the therapy system comprising:
a charged particle beam generator;
a beam transport system for transporting the charged particle beam;
an irradiation device for delivering the charged particle beam to the target volume; and
an energy-shaping device placed across a path of the charged particle beam, said energy-shaping device including:
a first pre-defined group of neighbouring energy-shaping elements that is adapted to deliver a first desired particle energy distribution at an output of said first pre-defined group of energy-shaping elements when crossed by particles of the charged particle beam; and
at least a second pre-defined group of neighbouring energy-shaping elements which is adapted to deliver a second desired particle energy distribution at an output of said second pre-defined group of energy-shaping elements when crossed by particles of the charged particle beam, said second desired particle energy distribution being different from said first desired particle energy distribution,
wherein each energy-shaping element of each of the first and second pre-defined groups of energy-shaping elements includes an individual layer of fluid or of a solid material, and
wherein the therapy system further includes a control unit which is configured:
to adjust the thickness of each fluid or solid material of each individual layer of fluid or of solid material of the energy-shaping elements of the first pre-defined group of neighbouring energy-shaping elements to obtain said first desired particle energy distribution when the irradiation device is oriented to deliver the particle beam to the target volume according to a first main beam direction, and
to adjust the thickness of each fluid or solid material of each individual layer of fluid or of solid material of the energy-shaping elements of the second pre-defined group of neighbouring energy-shaping elements to obtain said second desired particle energy distribution when the irradiation device is oriented to deliver the particle beam to the target volume according to the first main beam direction,
the said thickness of each fluid or of solid material being a thickness in a propagation direction of the charged particles of the charged particle beam.

2. The therapy system according to claim 1, wherein:
the first desired particle energy distribution comprises a first particle ratio (PRmin1) at a first minimum energy (Emin1) and a second particle ratio (PRmax1) at a first maximum energy (Emax1),
the second desired particle energy distribution comprises a third particle ratio (PRmin2) at a second minimum energy (Emin2) and a fourth particle ratio (PRmax2) at a second maximum energy (Emax2), and
Emax1 is different from Emax2.

3. The therapy system according to claim 2, wherein PRmax1 is different from PRmax2.

4. The therapy system according to claim 2, wherein Emin1 is different from Emin2.

5. The therapy system according to claim 4, wherein PRmin1 is different from PRmin2.

6. The therapy system according to claim 2, (Emax1−Emin1) is different from (Emax2−Emin2).

7. The therapy system according to claim 1, wherein each energy-shaping element has a cylindrical surface.

8. The therapy system according to claim 7, wherein all energy-shaping elements have the same hexagonal cross section.

9. The therapy system according to claim 1, wherein each energy-shaping element is a tube containing the fluid or the solid material.

10. The therapy system according to claim 1, wherein the energy-shaping elements are aligned with a propagation direction of the particles of the charged particle beam that cross them.

11. The therapy system according to claim 1, wherein each group of energy-shaping elements is aligned with respect to a propagation direction Z1x, Z2x, Z3x) of the particles of the incident particle beam.

12. The therapy system according to claim 1, wherein the therapy system includes beam scanner to scan the charged particle beam over the target volume, and in that a spot size of the charged particle beam in front of the energy-shaping device is substantially equal to the cross section of the first pre-defined group of neighbouring energy-shaping elements and substantially equal to the cross section of the second pre-defined group of neighbouring energy-shaping elements.

13. The therapy system according to claim 1, wherein the energy-shaping elements are arranged transversely with respect to a propagation direction of the particles of the charged particle beam.

14. The therapy system according to claim 1, wherein the charged particle beam generator is a cyclotron or a synchrotron.

15. The therapy system according to claim 14, wherein a nominal beam energy at an output of the charged particle beam generator is in the range of 70 MeV to 250 MeV.

16. The therapy system according to claim 13, wherein the energy-shaping elements are arranged perpendicularly with respect to a propagation direction of the particles of the charged particle beam.
